SEQUENCE LISTING (15 sequences)

SEQ ID NO: 1 - DNA, 314 bp, Homo sapiens
  modified_base (246)..(253): a, t, c, g, other or unknown
ccagtggtatagaaatcttcgatttttaaattcttaattttaggttgcagtttcatcactgtctgcggtaatcaagtttttagaactcttatcagatgattccaactttggacagtttgaactgactacttttgacttcagccagtatatgaaattggatattgcagcagtcagagcccttaacctttttcaggtaaaaaaaaaaaaaaaaaaaaaaaaaagggttaaaaatgttgattggttaannnnnnnngacagatagtgaagaaggcttagaaaggagctaaaagagttcgacatcaatattagacaag

SEQ ID NO: 2 - DNA, 324 bp, Homo sapiens
ggaaaagtattccagattgcctttctgtctggaggccatattattcatttattctctttctctctctccctctcccctcgcttccaatgaatctcttcactctaggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaaggtgagttttcttctcttaagggtgtggg

SEQ ID NO: 3 - DNA, 32 bp, Artificial Sequence
  Description of Artificial Sequence Synthetic probe
ggtaaaaaaaaaaaaaaaaaaaaaaaaaaggg

SEQ ID NO: 4 - DNA, 16 bp, Artificial Sequence
  Description of Artificial Sequence Synthetic probe
ggcttttttttttcct

SEQ ID NO: 5 - DNA, 21 bp, Artificial Sequence
  Description of Artificial Sequence Primer
tgcagtttcatcactgtctgc

SEQ ID NO: 6 - DNA, 21 bp, Artificial Sequence
  Description of Artificial Sequence Primer
tgcactcatcagagctacagg

SEQ ID NO: 7 - DNA, 10 bp, Artificial Sequence
  Description of Artificial Sequence Synthetic oligonucleotide
aaaaaaaaaa

SEQ ID NO: 8 - DNA, 9 bp, Artificial Sequence
  Description of Artificial Sequence Synthetic oligonucleotide
aaaaaaaaa

SEQ ID NO: 9 - DNA, 10 bp, Artificial Sequence
  Description of Artificial Sequence Synthetic oligonucleotide
tttttttttt

SEQ ID NO: 10 - DNA, 19 bp, Artificial Sequence
  Description of Artificial Sequence Primer
attggatattgcagcagtc

SEQ ID NO: 11 - DNA, 22 bp, Artificial Sequence
  Description of Artificial Sequence Primer
aaccaatcaacatttttaaccc

SEQ ID NO: 12 - DNA, 90 bp, Artificial Sequence
  Description of Artificial Sequence Primer
attggatattgcagcagtcagagcccttaacctttttcaggtaaaaaaaaaaaaaaaaaaaaaaaaaagggttaaaaatgttgattggtt

SEQ ID NO: 13 - DNA, 19 bp, Artificial Sequence
  Description of Artificial Sequence Primer
gaagatgctgcttctccaa

SEQ ID NO: 14 - DNA, 19 bp, Artificial Sequence
  Description of Artificial Sequence Primer
atcagagctacaggaacac

SEQ ID NO: 15 - DNA, 84 bp, Artificial Sequence
  Description of Artificial Sequence Primer
gaagatgctgcttctccaaagtgcattatgaaggaaaaaaaaaagcctggtgagactttcttcatgtgttcctgtagctctgat

(19) United States
(12) Patent Application Publication
(10) Pub. No.: US 2004/0091905 A1
(43) Pub. Date: May 13, 2004

(54) Method for detecting mutated polynucleotides within a large population of wild-type polynucleotides

(76) Inventor: Baochuan Guo, Solon, OH (US)

Correspondence Address: CALFEE HALTER & GRISWOLD, LLP, 800 SUPERIOR AVENUE, SUITE 1400, CLEVELAND, OH, 44114, US

(21) Appl. No.: 10/611,629

(22) Filed: Jul. 1, 2003

Related U.S. Application Data
(60) Provisional application No. 60/392,251, filed on Jul. 1, 2002.

(51) Int. Cl. C12Q 1/68; C12P 19/34

(52) U.S. Cl. 435/6; 435/91.2

(57) ABSTRACT

Methods are provided for detecting a mutant polynucleotide in mixture of mutant polynucleotides, wild-type polynucleotides and unrelated polynucleotides. The method uses an extension primer complementary to a first target sequence in both the wild-type and mutant polynucleotides. The method also uses a probe complementary to a second target sequence in the wild-type polynucleotides but not in the mutant polynucleotides. Extension of the primers annealed to the first target sequence in mutant polynucleotides produces long extension products. Extension of the primers annealed to the first target sequence in wild-type polynucleotides is blocked by the probe annealed to the second target sequence. Short extension products or no extension products are produced. The extension products are isolated and used in a polymerase chain reaction (PCR). The PCR preferentially amplifies long extension products.

This application claims priority from U.S. Provisional Patent Application Serial No. 60/392,251, filed on Jul. 1, 2002, which is incorporated herein by reference.

This invention was made, at least in part, with government support under National Institutes of Health Grants HG01815 and CA81653. The U.S. government has certain rights in the invention.

### FIELD OF THE INVENTION

The invention relates to a method for detecting a small number of mutant polynucleotides within a large number of wild-type polynucleotides within a larger background of unrelated polynucleotides. Specifically, the invention relates to a method for detecting a mutant microsatellite, indicative of cancer, in a sample of genome DNA from an individual also containing wild-type microsatellites.

### BACKGROUND

Microsatellites are short tracts of repeated nucleotides in the genomes of animals. The nucleotide sequences of wild-type microsatellites sometimes are found to contain small mutations (e.g., nucleotide deletions, insertions or substitution mutations). Such microsatellites are called mutant microsatellites and have a nucleotide sequence different than wild-type microsatellites. In humans, at least some of the mutations in microsatellites are associated with specific diseases, one being cancer. One example is certain types of colorectal cancer. Ten to fifteen percent of individuals with colorectal cancer have cells containing mutations within microsatellites. These mutations generally occur during DNA replication because polymerases often make mistakes in copying the repeats within microsatellites. Most often, nucleotide bases are deleted from microsatellites when the mistakes are made. In noncancerous cells, such mutations are normally corrected by postreplication mismatch repair mechanisms. In the colorectal cancer cells, however, mutations in DNA mismatch repair genes often prevent correction of the microsatellite mutations. In these cells, the microsateilite mutations become fixed in the genome and detection of the mutations can be diagnostic for the presence of colorectal cancer cells within an individual. Such colorectal cancers are called microsatellite instability (MSI) cancers. Other cancers, such as certain endometrial and gastric cancers, are also MSI cancers.

Screening for MSI cancers, based on detection of mutant microsatellites in cell samples from individuals is difficult because the mutant microsatellites from cancer cells are often significantly outnumbered by wild-type microsatellites from a large number of noncancerous cells in the samples. Additionally, both the mutant and wild-type microsatellites are present in a large background of unrelated polynucleotides from total genome DNA. Existing methods for detecting mutant microsatellites lack sensitivity and often lead to false-negative results (i.e., failure to detect mutant microsatellites that are present). Therefore, ideal screening assays have high sensitivity for mutant microsatellites, and also a low rate of false-positive results (i.e., detection of error-containing microsatellites when none are present).

One existing screening method for MSI cancers is a primer extension method designed to extend a primer by polynucleotide synthesis using mutant and wild-type microsatellites in a genome DNA sample from an individual as templates. Detection of primer extension products that are shorter than full length indicates the presence of microsatellites containing deletion mutations, that are indicative of cancer. The primer extension method, however, is not sensitive enough to detect the presence of small, early-stage colorectal cancers, where the abundance of mutant microsatellites in cell samples from individuals is very low. The method also has difficulty in detecting relatively small deletions within microsatellites. Additionally, the method typically uses a radiolabel, which is difficult to implement in automated methods.

Another existing screening method for MSI cancers is a polymerase chain reaction (PCR) method where PCR primers designed to anneal to target sequences on either side of a specific microsatellite are used to amplify the microsatellite. The PCR reaction also contains a peptide nucleic acid (PNA) probe that blocks amplification of wild-type microsatellites but not amplification of mutant microsatellites. In this method, the presence of a PCR amplification product indicates the presence of mutant microsatellites in the sample from the individual. The PCR method, however, lacks sensitivity and is prone to false-negative results. False-positive results also occur and can possibly be explained because the probe blocks polynucleotide synthesis from only one of the two DNA strands of the wild-type microsatellite template. Polymerase mistakes made during polynucleotide synthesis using a DNA strand that is not blocked as template, can lead to PCR amplification products, even though the sample from the individual contained no mutant microsatellites.

There is a need for new, highly sensitive methods having a low false-positive error rate, for detecting a small number of mutant microsatellites within a large number of wild-type microsatellites, both the microsatellites being present within a larger background of unrelated polynucleotides. Such methods are useful for screening individuals for the presence of colorectal cancer. Such methods may be more generally useful for detecting rare mutant polynucleotides within a mixture containing a large number of wild-type polynucleotides, normally within a larger background of unrelated polynucleotides.

### SUMMARY OF THE INVENTION

The present invention provides a method for detecting a small number of polynucleotides containing a mutation (i.e., mutant polynucleotides) within a mixture of mutant polynucleotides, a larger number of wild-type polynucleotides, and a still larger number of unrelated polynucleotides. The method uses a probe that is complementary to a region of the wild-type polynucleotides that corresponds to a region of the mutant polynucleotides that contains the mutation. The probe, therefore, is complementary to a nucleotide sequence in wild-type polynucleotides, but not to a nucleotide sequence in mutant polynucleotides. The method also uses an extension primer that is complementary to another region, present in both the wild-type and mutant polynucleotides, that in the wild-type polynucleotides, is present on the same polynucleotide strand as the region to which the probe is complementary, but which is located 5′ or upstream of the region to which the probe is complementary.

The first step of the method is to contact the probe with the polynucleotides under conditions in which the probe anneals to the region of the wild-type polynucleotides containing the complementary nucleotide sequence, but is less likely to anneal to the corresponding region, that contains the mutation, in the mutant polynucleotides. In the second step, the extension primer is contacted with the polynucleotides under conditions in which the extension primer anneals to its complementary region in both the wild-type and mutant polynucleotides. In the third step, the polynucleotides are contacted with a polymerase and nucleoside triphosphates under conditions where polynucleotide synthesis extends the extension primers, using the wild-type and mutant polynucleotides as templates, to produce extension products. In third step, polynucleotide synthesis that extends the extension primers annealed to wild-type polynucleotides is blocked by the probe annealed to the wild-type polynucleotides at a location 3′ or downstream of the extension primer. Polynucleotide synthesis that extends the extension primers annealed to mutant polynucleotides is not blocked. Therefore, polynucleotide synthesis using wild-type polynucleotides as templates predominantly produces extension products that are shorter in length (i.e., short extension products) than are extension products produced using mutant polynucleotides as templates (i.e., long extension products). In the fourth step, the extension products are isolated. In the fifth step, the isolated extension products are used as templates in a polymerase chain reaction (PCR) which preferentially amplifies the long extension products. In the sixth step, the products from the PCR are analyzed based on their size.

### BRIEF DESCRIPTION OF THE DRAWINGS

The present invention may be more readily understood by reference to the following drawings wherein:

FIG. 1. Schematic illustration of the PCPE-PCR principle of detecting mutant DNA (A)₉ in the presence of a large background of normal DNA (A)₁₀. The (A)₁₀ sequence is SEQ ID NO. 7. The (A)₉ sequence is SEQ ID NO. 8. The (T)₁₀ sequence is (SEQ ID NO. 9)

FIG. 2. TGF-β RII spectra obtained using different conditions. In this figure: i) PCR stands for use of PCR only; ii) PE-PCR denotes the use of primer extension (no probes) followed by PCR; iii) the percentage indicates the abundance of mutant DNA in the sample; and iv) the peaks labeled “A9” correspond to mutant DNA, and the peaks labeled “A10” correspond to wild-type DNA.

FIG. 3. TGF-β RII spectra obtained from three different samples using PCPE-PCR as follows: A) 0.1 ng of mutant DNA in 50 ng of wild-type DNA; B) 2 ng of mutant DNA in 1 μg of wild-type DNA; and C) 50 ng of wild-type DNA only.

FIG. 4. BAT26 spectra obtained from different conditions and samples. In this figure: i) PCR stands for the use of PCR only; ii) PE-PCR denotes the use of primer extension (no probes) followed by PCR; iii) the percentages indicate the abundance of mutant DNA in the sample; and iv) the numbers 86, 80, 79 and 74 specify the size of the corresponding PCR products.

FIG. 5. Nucleotide sequence of a part of wild-type BAT26 (GenBank Accession No. U41210) (SEQ ID NO. 1).

FIG. 6. Nucleotide sequence of a part of wild-type TGF-β RII which includes the (A)₁₀ sequence (GenBank Accession No. U52242) (SEQ ID NO. 2).

### DETAILED DESCRIPTION OF THE INVENTION

Definitions

Herein, “wild-type” polynucleotide, means a polynucleotide that has a nucleotide sequence considered to be normal or unaltered. In referring to a polynucleotide which is a microsatellite, wild-type refers to the nucleotide sequence of the particular microsatellite that is present in normal cells (noncancerous) of an individual.

Herein, “mutant” polynucleotide, means a polynucleotide that has a nucleotide sequence that is different than the nucleotide sequence of a wild-type polynucleotide. The difference in the nucleotide sequence of the mutant polynucleotide as compared to the wild-type polynucleotide is referred to as the mutation. The mutation is in the mutant polynucleotide.

Herein, “unrelated polynucleotide,” refers to polynucleotides that do not have nucleotide sequences in common (e.g., greater than 10 consecutive nucleotides in length) with either wild-type or mutant polynucleotides.

Herein, “anneal,” refers to nucleotides of a first single-stranded polynucleotide forming hydrogen bonds with complementary nucleotides of a second single-stranded polynucleotide.

Herein, “first target sequence,” refers to a nucleotide sequence within both mutant and wild-type polynucleotides to which an extension primer anneals.

Herein, “extension primer,” refers to a polynucleotide that is complementary to the first target sequence. The extension primer is capable of annealing to the first target sequence and acting as a primer for polynucleotide synthesis using either the wild-type or mutant polynucleotides as templates.

Herein, “corresponding sequence,” refers to a nucleotide sequence within the mutant polynucleotide that contains the mutation. This nucleotide sequence is said to “correspond” to the second target sequence, defined below.

Herein, “second target sequence,” is a nucleotide sequence within the wild-type polynucleotide that, except for the mutation, has the same nucleotide sequence as the corresponding sequence.

Herein, “probe,” refers to a polynucleotide that is complementary to the second target sequence. The probe is capable of annealing to the second target sequence and blocking polynucleotide synthesis that extends the extension primer, using the wild-type polynucleotide as a template.

The invention provides a method for detecting a mutant polynucleotide of low abundance in a population or mixture containing mutant polynucleotides, wild-type polynucleotides and, generally, a larger background of unrelated polynucleotides. The method is particularly useful for detecting a mutant microsatellite in a genome DNA sample from an individual, which also contains wild-type microsatellites.

Polynucleotides

Herein, polynucleotides are linear DNA molecules of various lengths. Polynucleotides can be from approximately 25 nucleotides in length to many kilobases in length. Polynucleotides can be single-stranded or double-stranded. The inventive method is used to detect single-stranded polynucleotides. However, the single-stranded polynucleotides that are detected by the methods of the present invention can be present as one strand of a double-stranded polynucleotide. The methods provide for denaturing the strands of a double-stranded polynucleotide so that the resulting single-stands can be detected.

The inventive method is designed to detect polynucleotides that have one or more mutations, called mutant polynucleotides, in a population or mixture of polynucleotides that do not have mutations, called wild-type polynucleotides. The mutant and wild-type polynucleotides are related, but not identical, in nucleotide sequence. The mutant and wild-type polynucleotides differ from each other by at least one nucleotide. Generally, the nucleotide differences between the mutant and wild-type polynucleotides are more than one nucleotide, although there must be some nucleotide sequence identity between the mutant and wild-type polynucleotides (i.e., the first target sequence), as is discussed below. The nucleotide differences can include nucleotide deletions, insertions and substitution mutations. When referring to a mutant polynucleotide, the nucleotides in the mutant polynucleotide that are different from nucleotides in the wild-type polynucleotide are called mutations. The region of the wild-type polynucleotide that, in the mutant polynucleotide, contains the mutation, is called the second target sequence. The region of the mutant polynucleotide that contains the mutation is called the corresponding region, because this region that contains the mutation corresponds to the region in the wild-type polynucleotide that does not contain the mutation. The inventive method uses the differences in nucleotide sequence between the second target sequence and the corresponding sequence to detect the mutant polynucleotides. Generally, these differences comprise less than 100 nucleotides. Generally, the mutations are known in order to use the inventive method.

It should be noted that, in addition to the differences in nucleotide sequence between mutant and wild-type polynucleotides that are within the region containing the corresponding sequence, there may be other differences between the mutant and wild-type polynucleotides that are present outside of the corresponding sequence. These additional nucleotide differences can be present anywhere within the mutant polynucleotide as compared to the wild-type polynucleotide, except within a region of both the mutant and wild-type polynucleotides that contains the first target sequence. Generally, these additional nucleotide differences are present upstream or 5′ of the first target sequence or downstream or 3′ of the second target sequence or corresponding sequence. The first target sequence is discussed in more detail later. Often, the additional nucleotide differences between mutant and wild-type polynucleotides are present at one or both ends of the polynucleotides. For example, if the mutant and wild-type polynucleotides come from genome DNA, it is likely not only that the lengths of the wild-type polynucleotides are different from the lengths of the mutant polynucleotides, but it is likely that one wild-type polynucleotide is different in length than another wild-type polynucleotide. Similarly, it is likely that one mutant polynucleotide is different in length than another mutant polynucleotide. The reason for this is because when genome DNA is isolated from cells, the break points within the DNA that give rise to polynucleotides is random. Polynucleotides of different lengths, as above, can be used in the inventive method. The method does not require mutant and wild-type polynucleotides of identical length. The method does not require all mutant polynucleotides to be the same length or all wild-type polynucleotides to be the same length. The method uses wild-type polynucleotides that contain both a first target sequence and a second target sequence, and uses mutant polynucleotides that contain both a first target sequence and a corresponding sequence.

Generally, the mutant polynucleotides are less frequent than the wild-type polynucleotides in the mixture of polynucleotides that is used in the inventive method. Generally, the mixture that contains the mutant polynucleotides and wild-type polynucleotides also contains a larger number of unrelated polynucleotides. Unrelated polynucleotides generally are polynucleotides that have large differences in nucleotide sequence as compared to either mutant or wild-type polynucleotides. Particularly, unrelated polynucleotides do not have nucleotide sequences identical to both the first target sequence and the second target sequence or the corresponding sequence.

In one embodiment of the method, the polynucleotides are microsatellites and the method detects mutant microsatellites in a mixture of mutant microsatellites, wild-type microsatellites and unrelated DNA which is genome DNA. A variety of different microsatellites are known. Some of these, for example, are BAT26, TGF-β RII (A)₁₀, NR-21, BAT25, D5S346, D2S123 and D17S250. Some other genes containing or associated with microsatellites include IGF2R, PTEN, transcription factors E2F4 and TCF4, apoptosis-associated genes BAX and caspace-5, mismatch-repair related genes MSH3, MSH6 and MBD4, WNT signaling-related genes AXIN2 and WISP3, and homeobox gene CDX2, and others.

As discussed, microsatellites are short tracts of repeated nucleotides found in animal genomes. Mutations within some microsatellites are associated with MSI cancers. For example, mutations that alter the nucleotide sequence of wild-type BAT26 microsatellites are frequently found in colorectal cancer. Mutations in a region of the TGF-β RII that has the sequence (A)₁₀ are found in 90% of colorectal cancers. The TGF-β RII mutations are generally changes within the (A)₁₀ sequence of the microsatellite. Another microsatellite, NR-21, contains an (A)₂₁ nucleotide sequence that contains an average deletion of (A)_{7.4} in certain colorectal cancers.

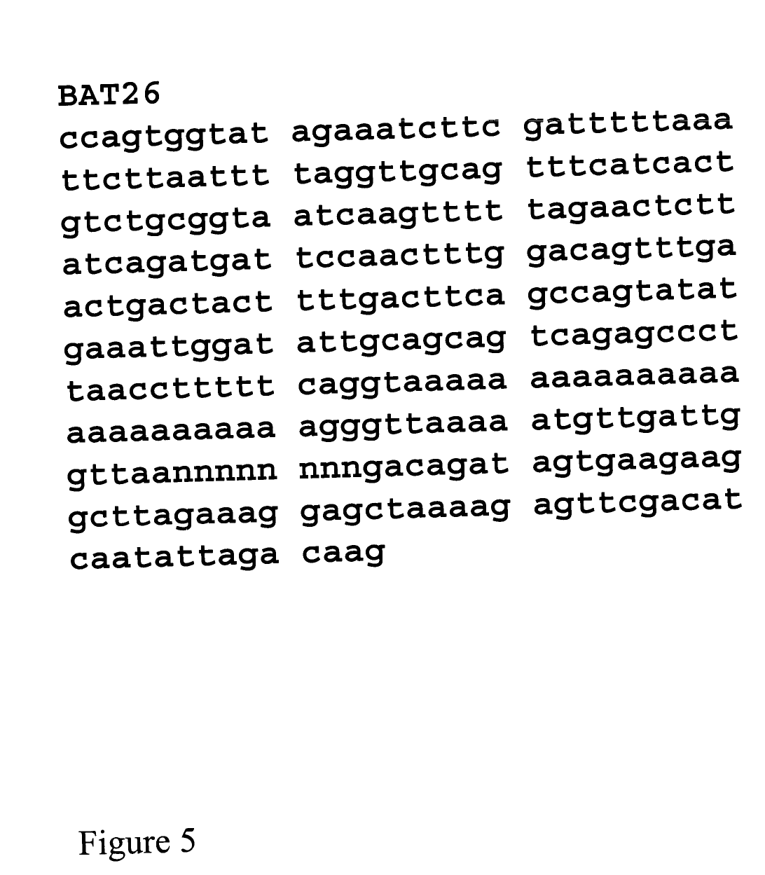

The nucleotide sequence in the human genome which includes wild-type BAT26 (GenBank Accession No. U41210) (SEQ ID NO. 1) is in exon 5 of the human mutator hMSH2 gene, is shown below and in FIG. 5:

| | |
|---|---|
| 1 | CCAGTGGTAT AGAAATCTTC GATTTTTAAA TTCTTAATTT TAGGTTGCAG TTTCATCACT |
| 61 | GTCTGCGGTA ATCAAGTTTT TAGAACTCTT ATCAGATGAT TCCAACTTTG GACAGTTTGA |
| 121 | ACTGACTACT TTTGACTTCA GCCAGTATAT GAAATTGGAT ATTGCAGCAG TCAGAGCCCT |
| 181 | TAACCTTTTT CAGGTAAAAA AAAAAAAAAA AAAAAAAAAA AGGGTTAAAA ATGTTGATTG |
| 241 | GTTAANNNNN NNNGACAGAT AGTGAAGAAG GCTTAGAAAG GAGCTAAAAG AGTTCGACAT |
| 301 | CAATATTAGA CAAG |

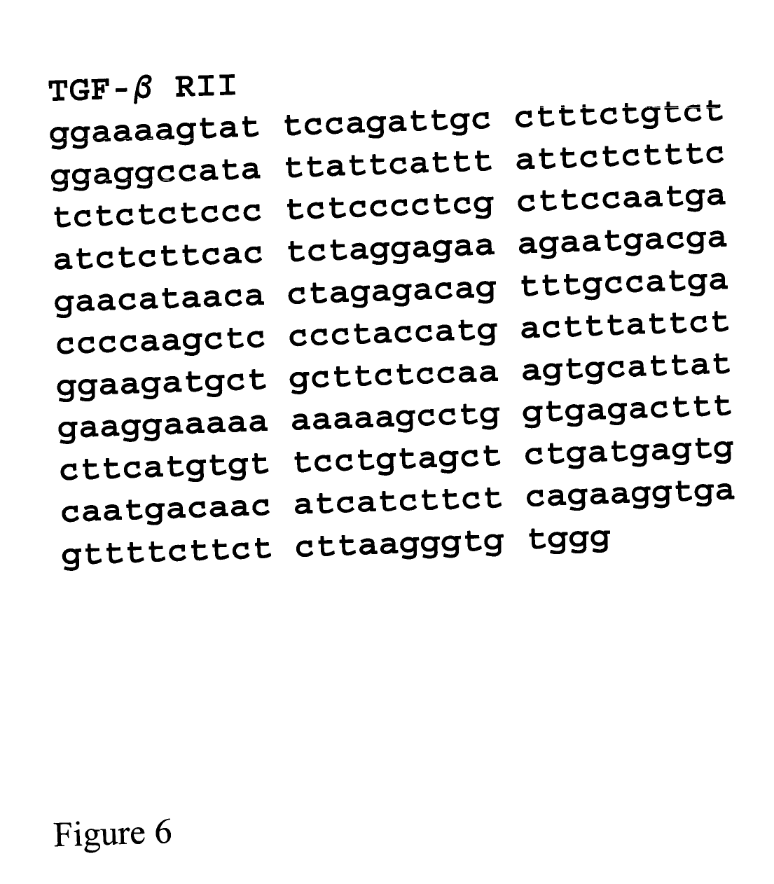

The nucleotide sequence in the human genome which includes wild-type TGF-β RII (A)₁₀ (GenBank Accession No. U52242) (SEQ ID NO. 2) is in exon 3 of the human transforming growth factor-beta type II receptor gene, is shown below and in FIG. 6.

| | |
|---|---|
| 1 | GGAAAAGTAT TCCAGATTGC CTTTCTGTCT GGAGGCCATA TTATTCATTT ATTCTCTTTC |
| 61 | TCTCTCTCCC TCTCCCCTCG CTTCCAATGA ATCTCTTCAC TCTAGGAGAA AGAATGACGA |
| 121 | GAACATAACA CTAGAGACAG TTTGCCATGA CCCCAAGCTC CCCTACCATG ACTTTATTCT |
| 181 | GGAAGATGCT GCTTCTCCAA AGTGCATTAT GAAGGAAAAA AAAAAGCCTG GTGAGACTTT |
| 241 | CTTCATGTGT TCCTGTAGCT CTGATGAGTG CAATGACAAC ATCATCTTCT CAGAAGGTGA |
| 301 | GTTTTCTTCT CTTAAGGGTG TGGG |

Design of Probes

To use the inventive method, an extension primer is designed to provide for linear amplification of both the mutant and wild-type polynucleotides by primer extension. A probe is also designed to provide for blocking of primer extension of the wild-type polynucleotides, but not for blocking of primer extension of the mutant polynucleotides. Design of the probe uses knowledge of one or more mutations that makes the nucleotide sequence of the mutant polynucleotide different from the nucleotide sequence of the wild-type polynucleotide. Generally the mutation is known. This means that the nucleotide sequence of the region of the mutant polynucleotide that contains the mutation (i.e., a region containing the corresponding region) is known, and the nucleotide sequence of the same region of the wild-type polynucleotide (i.e., a region containing the second target sequence) is also known. The probe is designed to be complementary to the first target sequence. The probe is not complementary to the corresponding sequence.

One example of a region containing a second target sequence and a region containing a corresponding region can be described using the TGF-β RII microsatellite. The TGF-β RII microsatellite contains the (A)₁₀ (SEQ ID NO. 7) nucleotide sequence in the wild-type microsatellite. Therefore, a continuous nucleotide sequence from the TGF-β RII microsatellite that contains the (A)₁₀ (SEQ ID NO. 7) sequence is considered to be a second target sequence. There are many different second target sequences possible. The (A)₁₀ (SEQ ID NO. 7) sequence of the TGF-β RII microsatellite can contain deletions when the microsatellite is mutated. In one case, the deletion can be a deletion of one A nucleotide. In this case, the mutant microsatellite contains an (A)₉ (SEQ ID NO. 8) sequence. A nucleotide sequence that is the same as the above described second target sequence (i.e., “corresponds” to the second target sequence), except that the (A)₁₀ (SEQ ID NO. 7) sequence is replaced by an (A)₉ (SEQ ID NO. 8) sequence, is considered to be a corresponding sequence. It can be said that the second target sequence in the wild-type polynucleotide is located in the same region of the wild-type polynucleotide that contains the corresponding sequence in the mutant polynucleotide. Similarly, it can be said that the corresponding sequence in the mutant polynucleotide is located in the same region of the mutant polynucleotide that contains the second target sequence in the wild-type polynucleotide.

Once the nucleotide sequences of the region containing the second target sequence and the region containing the corresponding sequence are known, these regions are used to design a probe, also called a blocking probe, to be used in the inventive method. A probe is a single-stranded polynucleotide designed to have a nucleotide sequence fully complementary to the second target sequence. “Fully complementary” means that every nucleotide within the probe sequence can form a hydrogen bond with its complementary nucleotide in the sequence of the wild-type polynucleotide (i.e., the second target sequence), with no mismatches. The second target sequence is not present in the mutant polynucleotide. Rather, the mutant polynucleotide contains the corresponding sequence, which because it contains the mutation, is different in nucleotide sequence than the second target sequence present in the wild-type polynucleotide. Because the nucleotide sequence of the second target sequence and the corresponding sequence are different, the probe is not fully complementary to a nucleotide sequence in the mutant polynucleotide.

When a first single-stranded polynucleotide sequence is able to form hydrogen bonds with a second single-stranded polynucleotide sequence, the first polynucleotide is said to have “annealed” to the second sequence. The annealed polynucleotides are said to have formed a “duplex.” A duplex is at least partially double-stranded. Although it may be possible for two polynucleotides that are not fully complementary to form a duplex, such a duplex is different from a duplex formed between two polynucleotides that are fully complementary. In general, every nucleotide within a polynucleotide that is fully complementary to another polynucleotide forms one or more hydrogen bonds with its complementary nucleotides in the other polynucleotide when a duplex is formed. In contrast, not every nucleotide within a polynucleotide that is not fully complementary forms hydrogen bonds with the other polynucleotide. The nucleotides that do not form hydrogen bonds are called “mismatched nucleotides” or “mismatches.” Duplexes between two fully complementary polynucleotides, in general, form more stable duplexes than do polynucleotides that form duplexes containing mismatches. Stability of duplexes refers to the temperature at which the hydrogen bonds formed between the two single-stranded polynucleotides are broken and the duplex becomes two single-stranded polynucleotides. The higher the temperature at which the hydrogen bonds are broken or “melted”, the more stable the duplex. Tₘ is a temperature measurement used to designate stability of duplexes. Tₘ is the temperature at which 50% of the hydrogen bonds comprising a duplex are broken. The higher the Tₘ for a duplex, the more stable is that duplex.

Tₘ can be calculated in a variety of ways. Since the thermal energy required to break hydrogen bonds between two nucleotides that form hydrogen bonds is known (e.g., A−T and G−C), the Tₘ for a duplex formed between two nucleotide sequences, at a specified salt concentration, can be calculated using methods known in the art. The Tₘ for a duplex can also be experimentally determined by a variety of methods. In one method, UV with a cell holder and a temperature station (Aglient) is used. In another method, a duplex between two polynucleotide sequences is incubated in a mixture also containing a dye such as SYBR Green I. The dye emits a fluorescence signal only in the presence of a duplex. As the temperature of the mixture is raised, the fluorescence signal is measured. At increasing temperatures, the Tₘ of the duplex is approached and then exceeded, and hydrogen bonds are broken or melted. As this occurs, emitted fluorescence of the dye decreases. Therefore, a plot of temperature versus emitted fluorescence signal is used to determine Tₘ.

Similarly, the Tₘ for annealing of the probe to the second target sequence, and to the corresponding sequence, can be determined. The Tₘ for annealing of the probe to the second target sequence in the wild-type polynucleotide is herein called the “second Tₘ.” The Tₘ for annealing of the probe to the corresponding sequence in the mutant polynucleotide is herein called the “third Tₘ.” The second Tₘ is higher than the third Tₘ, reflecting the increased stability of a duplex without mismatches (i.e., the probe annealing to the second target sequence) as compared to a duplex with mismatches (i.e., the probe annealing to the corresponding sequence). Preferably, the second target sequence is chosen such that the difference between the second Tₘ and the third Tₘ is maximized. That is, if probes of two different nucleotide sequences, that anneal to two different second target sequences, are made. Then, the probe where the difference between the second and third Tₘ's are greatest is preferably used. Different probes can be designed, for example, by changing the length of the probe, changing the second target sequence, or by changing the location within the probe where the mismatches occur when the probe anneals to the corresponding sequence.

Probes can be of a number of types. Generally, probes can be of any chemistry that can anneal and form a duplex with the polynucleotides. One type of probe is an oligonucleotide probe. Oligonucleotide probes generally can be between 15 and 50 nucleotides in length. Preferably, oligonucleotide probes are between 20 and 30 nucleotides in length. Preferably, oligonucleotide probes are designed in such a way that cleavage, by DNA polymerases for example, is minimized. One method of minimizing cleavage is to phosphorothioate the first 5 nucleotide positions at both the 5′ and 3′ ends of the oligonucleotide. Preferably, oligonucleotide probes are also designed in such a way that the ends of the probe cannot be extended by polynucleotide synthesis. One method for preventing extension of the probe by polynucleotide synthesis is to phosphorylate the 3′ nucleotide of the probe. Oligonucleotides are preferably used when it is desired to have a probe of a length greater than about 17 nucleotides.

Another type of probe is a peptide-nucleic acid probe (PNA). PNAs are DNA mimics in which the deoxyribose-phosphate backbone is replaced by an oligoamide consisting of N-(2-aminoethyl)glycine units. PNA mimics DNA in terms of its ability to recognize and anneal to complementary nucleic acid sequences but does so with higher thermal stability (Tₘ) and specificity than corresponding oligonucleotide probes. A single base mismatch in a PNA-DNA duplex is much more destabilizing than in the corresponding DNA-DNA duplex (i.e., creates a larger ΔTₘ than does a single base mismatch in a DNA-DNA duplex; meaning that the difference between the second Tₘ and the third Tₘ is larger with a PNA probe than with the same oligonucleotide probe). Furthermore, PNA cannot function as a primer for DNA polymerase (i.e., it cannot be extended by polynucleotide synthesis). PNAs generally cannot be made longer than 17 bases long, whereas oligonucleotides can be made much longer.

Probes can also be conformationally restricted DNA-analogues. One such DNA analogue is a locked nucleic acid (LNA). LNA's generally contain one or more 2′-O, 4′-C-methylene-β-D-ribofuranosyl nucleoside monomers. Other types of cnemistries can also be used to make the probes of the present invention.

Probes can also contain a variety of chemical groups such as phosphorylated groups and thiol groups. Probes can also contain attached molecules, such as biotin molecules, various dye molecules, and others.

In one embodiment, the probe for use in detection of mutant BAT26 microsatellites is an oligonucleotide of sequence 5′-GGTAAAAAAAAAAAAAAAAAAAAAAAAAAGGG-3′ (SEQ ID NO. 3). In another embodiment, the probe for use in detection of mutant TGF-β RII (A)₁₀ microsatellites is a PNA of sequence 5′-GGCTTTTTTTTTTCCT-3′ (SEQ ID NO. 4).

Design of Extension Primers

In addition to the probe, the inventive method also uses an extension primer. The extension primer acts as a primer for polynucleotide synthesis that extends the 3′ end of the extension primer using the wild-type and the mutant polynucleotide as templates, as is discussed in more detail below. The extension primer is a single-stranded polynucleotide designed so that it has a nucleotide sequence that is fully complementary to a region that contains a nucleotide sequence that is present in both the mutant and wild-type polynucleotides. The nucleotide sequence to which the extension primer is fully complementary is herein called the “first target sequence.” In the wild-type polynucleotide, the first target sequence is on the same polynucleotide strand as is the second target sequence and is located upstream or 5′ of the second target sequence. In the mutant polynucleotide, the first target sequence is on the same strand as is the corresponding sequence and is located upstream or 5′ of the corresponding sequence.

The distance between the first target sequence and the second target sequence in the wild-type polynucleotides, or between the first target sequence and the corresponding sequence in the mutant polynucleotides, can be variable. In one embodiment of the method, the distance can be as much as approximately 1000 nucleotides. In another embodiment of the method, there may be no nucleotides separating the first target sequence and the second target sequence/corresponding sequence. In one embodiment, the nucleotide sequence of the first target sequence partially overlaps with the second target sequence in wild-type polynucleotides and overlaps with the corresponding sequence in the mutant polynucleotides. Herein, “overlap” means that the nucleotide sequence of the first target sequence contains part of the nucleotide sequence of the second target sequence and corresponding sequence. The first target sequence, however, does not contain the complete nucleotide sequence of the second target sequence or corresponding sequence. To determine how much of the nucleotide sequence of the second target sequence and corresponding sequence can be contained in the first target sequence (i.e., to determine the extent of the overlap), the 5′ end of the second target sequence is aligned with the 5′ end of the corresponding sequence. Then, beginning at the 5′ ends, the identity of the aligned nucleotides is compared. At the ends of the two sequences, the nucleotides are identical. As the comparison moves toward the 3′ ends, there will be non-identity of nucleotides at the same position in the second target sequence compared to the corresponding sequence. The position where the non-identity occurs identifies the position of a mutation. The first target sequence can contain that part of the aligned second target sequence and corresponding sequence, from the 5′ end until, and not including, the position where there is non-identity (i.e., the position of the mutation). Therefore, the first target sequence does not contain that part of the second target sequence/corresponding sequence that identifies the position of the mutation.

Extension primers are preferably oligonucleotide primers and generally are between 10 to 30 nucleotides in length. Preferably, extension primers are between 18 to 22 nucleotides in length. The extension primers are long enough to prevent annealing to sequences other than the first target sequence in the wild-type and mutant polynucleotides. Extension primers with long runs of a single base should be avoided, if possible. Primers should preferably have a percent G+C content of between 40 and 60%. If possible, the percent G+C content of the 3′ end of the primer should be higher than the percent G+C content of the 5′ end of the primer. Extension primers should not contain nucleotide sequences that can anneal to another nucleotide sequence within the same or another extension primer.

The extension primer anneals to the first target sequence with a first Tₘ. The extension primer is chosen such that the first Tₘ is lower than the second Tₘ (the second Tₘ is the Tₘ for annealing of the probe to the second target sequence which is present in the wild-type polynucleotide), but higher the third Tₘ, (the third Tₘ is the Tₘ for annealing of the probe to the corresponding sequence in the mutant polynucleotide).

Extension primers may have modifications and/or additional molecules attached, as long as the 3′ end of the extension primer can be extended by polynucleotide synthesis. In one embodiment, the extension primer has one or more biotin molecules attached. Such biotin molecules are useful for isolating the extended primers using solid phase extraction methods, as are described in more detail below.

In one embodiment, the extension primer for detection of mutant BAT26 microsatellites is 5′-biotin-TGCAGTTTCATCACTGTCTGC-3′ (SEQ ID NO. 5). In another embodiment, the extension primer for detection of mutant TGF-β RII microsatellites is 5′-biotin-TGCACTCATCAGAGCTACAGG-3′ (SEQ ID NO. 6).

Input Polynucleotides

The mixture of mutant and wild-type polynucleotides, generally also containing unrelated polynucleotides, can come from a variety of sources.

In one embodiment, mutant polynucleotides and wild-type polynucleotides are obtained from different sources (e.g., two different cell lines), then are mixed to provide a sample that is used in the inventive method. Genome DNA is isolated from one cell line that provides mutant polynucleotides. Genome DNA is also isolated from another cell line that provides wild-type polynucleotides. Genome DNA is isolated from the cell lines using standard methods. The isolated genome DNAs are mixed in a known amount (see Example 1).

In another embodiment, genome DNA that contains wild-type polynucleotides and is suspected of additionally containing mutant polynucleotides is obtained from a human sample that contains cells. Such samples can come from blood, other bodily fluids, biopsy samples, and the like. One preferred human sample is a stool sample. Human stool samples contain human cells, including cells from colon and rectum from which genome DNA can be isolated. Human stools also contain impurities, including excessive amounts of bacteria, whose DNA can inhibit enzymatic reactions. It is preferable to remove such impurities from a genome sample that is used in the inventive method.

A variety of methods exist for isolating DNA (human genome DNA and bacterial DNA) from stools. In addition, commercially available kits exist for this purpose. One such commercial kit is the QIAamp® DNA Stool Mini Kit (QIAGEN Inc., Valencia, Calif.). It has been reported that at least 4000 copies of human genome DNA can be extracted from 10 g of stools. Such a yield results in 16 or more copies of a mutant polynucleotide if the abundance of a mutant microsatellite is 0.4%.

Additionally, human genome DNA from human cells in stool comprises a large fraction of bacterial DNA from bacterial cells in stool. It is preferable, therefore, to enrich the human genome DNA. It is more preferable to enrich the human genome DNA for the desired polynucleotides. One such method for enriching for specific nucleotide sequences is called “sequence specific hybrid capture.” In this method, one or more capture probes (can be oligonucleotides, PNAs, LNAs, etc.) of a nucleotide sequence complementary to the nucleotide sequence of the microsatellite that is desired to be enriched is used. Briefly, in one embodiment of the method, the DNA isolated from stool is mixed with an equal volume of 1-2 M NaCl serving as the buffer of both hybridization and bead capture. The DNA is denatured at 95° C., followed by incubation with the sequence-specific capture probes, which are biotinylated, at a temperature which allows annealing of the capture probes with the polynucleotides in the total stool DNA. Then, streptavidin-coated magnetic beads are added to and incubated with the DNA solution at room temperature. After incubation with the beads, the supernatant containing the DNA that has not annealed with the capture probes, is removed. The bead-capture probe complexes are washed, resuspended in buffer and then used in the PCPE procedure, as is described below.

It has been found that stools can be lysed above room temperature, yielding more DNA. Subsequent use of isolation methods, such as sequence-specific hybrid capture, can be used to then increase the microsatellites obtained.

PCPE

After the probe and extension primer have been designed and made, and after the input polynucleotides have been obtained, the first step of the inventive method is PCPE. PCPE is probe clamping primer extension. In PCPE, the input polynucleotides are contacted with the probe under conditions where the probe preferentially anneals with the second target sequence in the wild-type polynucleotides compared to the corresponding sequence in the mutant polynucleotides. “Preferential annealing” means contacting the probe with the mixture of polynucleotides at a Tₘ that is high enough to allow maximum duplex formation between the probe and the second target sequence in the wild-type polynucleotide, but that allows less than maximum duplex formation between the probe and the corresponding sequence in the mutant polynucleotide. As discussed earlier, the second Tₘ, which is the Tₘ for duplexes between the probe and second target sequence, is higher than the third Tₘ, which is the Tₘ for duplexes between the probe and the corresponding sequence. Preferential annealing occurs when the temperature at which the probe is contacted with the polynucleotides is a temperature equal to or less than the second Tₘ, but greater than the third Tₘ. Preferably, the temperature for preferential annealing is a temperature that is closer to the second Tₘ than to the third Tₘ.

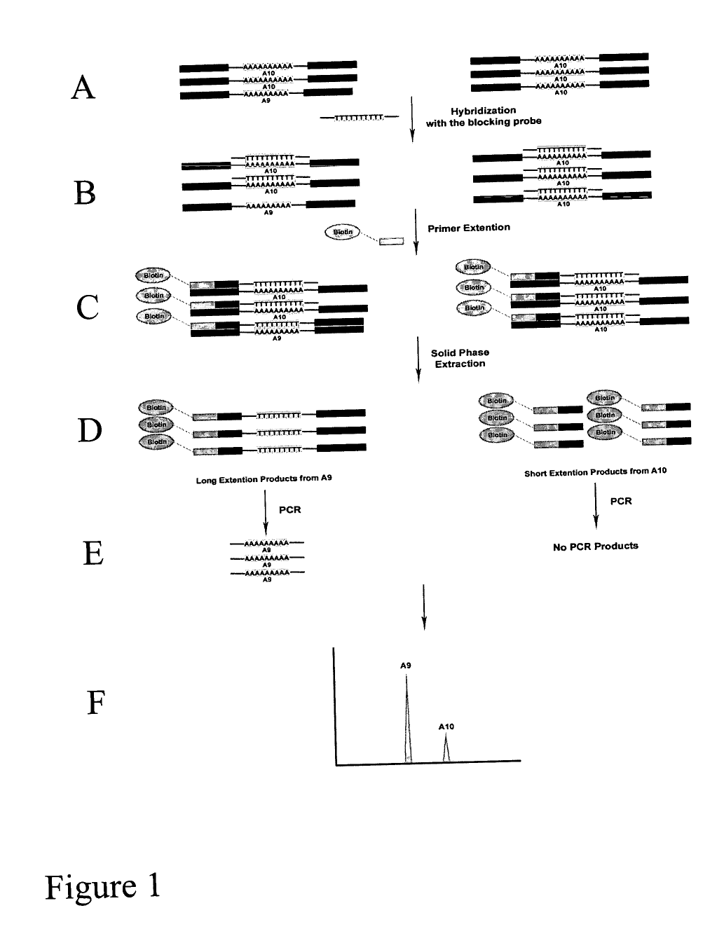

The above steps can be seen in a schematic diagram, that is shown in FIG. 1. In FIG. 1A, wild-type microsatellites are shown containing (A)₁₀ (SEQ ID NO. 7) and mutant microsatellites are shown containing (A)₉ (SEQ ID NO. 8). As shown in the diagram, the wild-type microsatellite is present in great excess as compared to the mutant microsatellite, as is expected in the case where genome DNA is obtained from a cell sample from an individual that contains a small number of cancerous cells and a large number of noncancerous cells. In FIG. 1B, a probe is shown as —TTTTTTTTTT—, or —(T)₁₀—(SEQ ID NO. 9). In the embodiment shown, the blocking probe contains not only a sequence of 10 T's, (T)₁₀ (SEQ ID NO. 9), but also contains nucleotide bases preceding the 10 T's and nucleotide bases following the 10 T's (represented by the dashes on either side of the (T)₁₀ in the diagram). The nucleotide bases in the blocking probe that precede and follow the 10 T's are chosen to be complementary to the corresponding nucleotide bases that flank the repeated T sequence in the genome. As shown in FIG. 1B, the probe, when added to the sample of microsatellites, anneals to the wild-type microsatellites, that contain (A)₁₀ (SEQ ID NO. 7), but do not anneal to the mutant microsatellites, here containing (A)₉ (SEQ ID NO. 8). In this particular example, the second target sequence contains (A)₁₀ (SEQ ID NO. 7). Since the corresponding sequence within the error-containing microsatellite contains (A)₉ (SEQ ID NO. 8), the mutant microsatellite does not have a second target sequence.

After annealing of the probe to the second target sequence has occurred, the extension primer is contacted with the polynucleotides under conditions which allow the extension primer to anneal with the first target sequence in both the mutant and wild-type polynucleotides. Such conditions are provided when the temperature is at or near the first Tₘ. At too high a temperature (e.g., a temperature significantly above the first Tₘ), the extension primer will not form a duplex with the first target sequence. At too low a temperature (e.g., a temperature significantly below the first Tₘ), the probe may form duplexes with the corresponding sequence.

In the case where there is overlap between the first target sequence and the second target sequence/corresponding sequence, the extension primer may not be able to anneal with the first target sequence under these conditions, due to the duplex between the probe and the second target sequence.

After annealing of the extension primer to the polynucleotides, a DNA polymerase and nucleoside triphosphates are contacted with the mixture under conditions where polynucleotide synthesis can occur. Such conditions are known in the art. Polynucleotide synthesis occurs by extending the 3′ end of the extension primer, if it has annealed to the first target sequence. The polynucleotide synthesis uses the wild-type polynucleotide as a template when an extension primer that has annealed to the wild-type polynucleotide is extended. The polynucleotide synthesis uses the mutant polynucleotide as a template when an extension primer that has annealed to the mutant polynucleotide is extended. The extension primers that have been extended by polynucleotide synthesis are called “extension products.” At some point, as polynucleotide synthesis extends the extension primer that has annealed to the wild-type polynucleotide, further extension will be blocked due to the probe that has annealed to the second target sequence. These extension products are called “short extension products.” Polynucleotide synthesis that extends the extension primer that has annealed to the mutant polynucleotide is not blocked because there is no probe annealed to the corresponding sequence in the mutant polynucleotide. These extension products are called “long extension products.” Long extension products have a longer length than short extension products.

The above steps can be seen schematically in FIG. 1C, which shows addition of an extension primer to the mixture. The extension primer is shown as a lightly-shaded box and, in this embodiment, has an attached biotin molecule. Also shown is the result of polynucleotide synthesis that extends the 3′ end of the extension primer, using the polynucleotides as templates. It can be seen from the diagram that the extension products produced from use of the wild-type, (A)₁₀ (SEQ ID NO. 7) polynucleotide as template are shorter (i.e., short extension products) than the extension products made from use of the mutant, (A)₉ (SEQ ID NO. 8) polynucleotide as template (i.e., long extension products), due to the probe annealed to the second target sequence in the wild-type polynucleotides. In a more general case, the above steps lead to enrichment of the long extension products with the mutant polynucleotides as template.

In the example shown in FIG. 1, there is no overlap between the first target sequence and the second target sequence/corresponding sequence. The effect is that both short and long extension products are made. In other embodiments, where there is such sequence overlap, short extension products may not be produced.

Isolation of Extension Products

After the PCPE reaction, the extension products are isolated from the reaction in which the PCPE occurred. Generally, this isolation step comprises enrichment of both long extension products, and short extension products if they are present, away from the wild-type, mutant and unrelated polynucleotides in the mixture. In other embodiments, however, it may be possible to isolate only the long extension products from the mixture.

One method for isolating the extension products from the mixture is a solid phase extraction method (see Example 3). In one type of solid phase extraction method, the biotin attached to the extension primer, the extension primer having been extended by polynucleotide synthesis into an extension product, is bound to streptavidin-coated beads, while the mutant, wild-type and unrelated polynucleotides are washed away. Briefly, the PCPE reaction mixture is heated to a temperature to denature DNA in the mixture (95° C.). The mixture is then rapidly cooled to 0° C. The mixture is then treated with streptavidin-coated beads that capture the biotinylated DNA molecules, followed by removing the supernatant containing the polynucleotides. An additional washing step by a buffer containing 0.05-0.1 M NaOH may be added as this further denatures genome DNA, thus removing the polynucleotides from the biotinylated DNA fragments. Then, the beads are washed a few times to remove remaining polynucleotides. The captured single strand-DNA fragments are separated from the beads by heating the beads and then are used in PCR, as described below. Kits for performing solid-phase extraction are commercially available. For example, Dynal uses a specific biotin-streptavidin binding buffer that improves capture of 1 kb DNA molecules.

FIG. 1D shows the results of isolating the extension products.

PCR

The isolated extension products are then used as templates in a PCR reaction, where the long extension products are preferentially amplified. Preferential amplification of long extension products herein means that there is more amplification of the long extension products than the short extension products in a PCR reaction. The basis for the preferential amplification is the longer length of the long extension product. Both the long extension products and short extension products have the same 5′ end. Because the long extension product is longer than the short extension product, there are nucleotide sequences at the 3′ end of the long extension product that are not present in the short extension product. A first PCR primer is, therefore, designed that is complementary to nucleotides in the 3′ end of the long extension product, that are not present in the short extension product. A second PCR primer is designed that is identical to a nucleotide sequence present in both the short and long extension products. Use of the first and second PCR primers in a PCR reaction results in amplification of the long extension product, while the short extension product is not amplified. The products of the PCR reaction are referred to as PCR products.

PCR primers normally are between 10 to 30 nucleotides in length and have a preferred length from between 18 to 22 nucleotides. PCR primers are also chosen subject to a number of other conditions. PCR primers should be long enough (preferably 10 to 30 nucleotides in length) to minimize hybridization to greater than one region in the template. Primers with long runs of a single base should be avoided, if possible. Primers should preferably have a percent G+C content of between 40 and 60%. If possible, the percent G+C content of the 3′ end of the primer should be higher than the percent G+C content of the 5′ end of the primer. Primers should not contain sequences that can anneal to another sequence within the primer (i.e., palindromes). Two primers used in the same PCR reaction should not be able to anneal to one another. Although PCR primers are preferably chosen subject to the recommendations above, it is not necessary that the primers conform to these conditions. Other primers may work, but have a lower chance of yielding good results.

PCR primers are preferably chosen using one of a number of computer programs that are available. Such programs choose primers that are optimum for amplification of a given sequence (i.e., such programs choose primers subject to the conditions stated above, plus other conditions that may maximize the functionality of PCR primers). One computer program is the Genetics Computer Group (GCG recently became Accelrys) analysis package which has a routine for selection of PCR primers. There are also several web sites that can be used to select optimal PCR primers to amplify an input sequence. One such web site is http://alces.med.umn.edu/rawprimer.html. Another such web site is http://www-genome.wi.mit.edu/cgi-bin/primer/primer3_www.cgi.

Once the first and second PCR primers are designed, they are mixed with the extension products and the PCR amplification reaction is performed. A standard PCR reaction contains a buffer containing 10 mM Tris-HCl (pH 8.3), 50 mM KCl, and 2.0 mM MgCl₂, 200 uM each of dATP, dCTP, dTTP and dGTP, two primers of concentration 0.5 uM each, 7.5 ng/ul concentration of template cDNA and 2.5 units of Taq DNA Polymerase enzyme (a PCR polymerase). Variations of these conditions can be used and are well known to those skilled in the art.

The PCR reaction is preferably performed under high stringency conditions. Such conditions are equivalent to or comparable to denaturation for 1 minute at 95° C. in a solution comprising 10 mM Tris-HCl (pH 8.3), 50 mM KCl, and 2.0 mM MgCl₂, followed by annealing in the same solution at about 62° C. for 5 seconds.

In one embodiment, long extension products from the BAT26 microsatellite are amplified using PCR. In this embodiment, the second PCR primer is 5′-D4-ATTGGATATTGCAGCAGTC-3′ (SEQ ID NO. 10), where D4 represents a fluorescent dye that can be detected using methods described below. The first PCR primer is 5′-AACCAATCAACATTTTTAACCC-3′ (SEQ ID NO. 11). The PCR product that results from amplification of the long extension product using these PCR primers is 5′-ATTGGATATTGCAGCAGTCAGAGCCCTTAACCTTTTTCAGGTAAAAAAAAAAAAAA AAAAAAAAAAAAGGGTTAAAAATGTTGATTGGTT-3′ (SEQ ID NO. 12).

In another embodiment, long extension products from the PCR primers for TGF-β RII microsatellite are amplified using PCR. In this embodiment, the second PCR primer is 5′-GAAGATGCTGCTTCTCCAA-3′ (SEQ ID NO. 13). The first PCR primer is 5′-D4-ATCAGAGCTACAGGAACAC-3′ (SEQ ID NO. 14). The PCR product that results from amplification of the long extension product using these PCR primers is 5′-GAAGATGCTGCTTCTCCAAAGTGCATTATGAAGGAAAAAAAAAAGCCTGGTGAGAC TTTCTTCATGTGTTCCTGTAGCTCTGAT-3′ (SEQ ID NO. 15).

FIG. 1E shows the results of such a PCR. As shown in the diagram, the result of the PCR is that the (A)₉ (SEQ ID NO. 8) microsatellite is amplified while little or no amplification of the (A)₁₀ (SEQ ID NO. 7) microsatellite occurs.

Analysis of PCR Products

The products of the PCR reaction generally are analyzed to determine the different sizes and/or abundance of PCR products that have been produced. Because the nucleotide sequence of the second target sequence in the wild-type polynucleotide, and the corresponding sequence in the mutant polynucleotide are known, it is possible to ascertain whether a PCR product of a given length is from a wild-type polynucleotide, a mutant polynucleotide, or from some other source, such as PCR slippage.

There are a variety of methods that can be used to determine the size and abundance of PCR products. One method is electrophoresis, preferably polyacrylamide or agarose gel electrophoresis. Using electrophoresis, the products of a PCR reaction are separated based on their size. Additional methods, such as densitometry, can be used to determine the amount or abundance of PCR product of each size.

Another method for determining the size and abundance of PCR products is DNA sequencing. In one embodiment, a CEQ8000 sequencer (Beckman Coulter, Fullerton, Calif.) ahs been used.

Another method for determining the size and abundance of PCR products is matrix-assisted laser-desorption/ionization time-of-flight (MALDI-TOF) mass spectrometry.

FIG. 1F shows a graph in which the relative amounts of the (A)₉ (SEQ ID NO. 8) and (A)₁₀ (SEQ ID NO. 7) extension products are shown.

Sensitivity of PCPR-PCR

At a minimum, the PCPE-PCR method detects mutant polynucleotides in a mixture that contains as little as 5 mutant polynucleotide molecules in a 500-fold excess of wild-type polynucleotide molecules (0.2% mutant). Five mutant polynucleotide molecules can be obtained from 10 g of stool.

Multiplexing

In one embodiment of the inventive method, the PCPE-PCR is used as a multiplexed assay. Multiplexed means that, instead of using PCPE-PCR to detect a single mutant polynucleotide, the PCPE-PCR assay is used to simultaneously detect more than one mutant polynucleotide in a mixture. There are a variety of multiplexed assays that can be used. For example, a multiplexed assay can be used to detect different mutant microsatellites from the same wild-type microsatellite. For example, a single PCPE-PCR assay could be used to detect mutant (A)₉ (SEQ ID NO. 8) and other sequences from the wild-type (A)₁₀ (SEQ ID NO. 7) microsatellite of TGF-β RII. Preferably, a multiplexed PCPE-PCR is used to detect different mutant microsatellites from different wild-type microsatellites. For example, a multiplexed PCPE-PCR assay could be used to detect mutant TGF-β RII (A)₁₀ microsatellites and mutant BAT26 microsatellites.

In such a multiplexed PCPE-PCR assay, the PCPE step contains an extension primer and a probe for each mutant polynucleotide that is being detected. Preferably, the first Tₘ for the different polynucleotides are similar, the second Tₘ for the different polynucleotides are similar. It is also preferable that a probe for one polynucleotide does not block extension of an extension primer from another polynucleotide. In the subsequent PCR step, the first and second PCR primer is used for each long extension product that is trying to be detected.

### EXAMPLES

The invention may be better understood by reference to the following examples, which serve to illustrate but not to limit the present invention.

### Example 1

### DNA Samples

In some studies, DNA containing known mutations in specific microsatellite alleles (i.e., error-containing satellites) was obtained from human cell lines and was mixed with normal human DNA containing wild-type sequences in the specific microsatellite alleles (i.e., wild-type microsatellites). Normal human DNA was purchased commercially (Sigma Chemical Co.; St. Louis, Mo.). DNA containing mutant TGF-β RII microsatellites was extracted from cell line HCL116. DNA containing mutant BAT26 microsatellites was extracted from cell lines HCL116, V481 and HECLA. DNA was extracted using standard methods. DNA samples containing a low abundance of mutant microsatellites and a high abundance of wild-type microsatellites were prepared by mixing small amounts of DNA isolated from the HCL116, V481 and HEC1A cell lines with larger amounts of normal human DNA. The abundance and number of mutant DNA molecules in the created samples were estimated based on the number of mutant DNAs in the original samples and dilution factors.

### Example 2

### Blocking Probes

A PNA probe of 5′-GGCTTTTTTTTTTCCT-3′ (SEQ ID NO. 4) (Applied Biosystems; Foster City, Calif.) was used for TGF-RII microsatellites. An oligonucleotide probe of 5′-GGTAAAAAAAAAAAAAAAAAAAAAAAAAGGG-3′ (SEQ ID NO. 3) was used for BAT26 microsatellites. The oligonucleotide probe was phosphorothioated at the first 5 positions at the 5′ and 3′ ends to minimize cleavage of the probe by DNA polymerases, and was also phosphorylated at the 3′ end to prevent the probe from undergoing primer extension.

### Example 3

### PCPE-PCR Applied to Short Microsatellite Sequences

PCPE-PCR was first used to detect mutations in short microsatellite sequences. Herein, short microsatellite sequences contain 12 or fewer repeats of a single nucleotide base, this sequence normally altered by 1-2 bases when mutated. In these studies, the TGF-β RII microsatellite was used which, in its wild-type form, contains (A)₁₀ (SEQ ID NO. 7). DNA from the HCL116 cell line has mutant TGF-β RII microsatellites containing (A)₉ (SEQ ID NO. 8).

PCPE was carried out in 25 μl reactions using 3 μM of the PNA blocking probe described in Example 2, 0.01 μM of the extension primer 5′-Biotin-TGCACTCATCAGAGCTACAGG-3′ (SEQ ID NO. 6), 0.1 μM each of nucleoside triphosphates dCTP, dTTP, dATP and dGTP, 2 mM of MgCl₂, 1X AmpliTag Gold® PCR buffer and 0.5 units of AmpliTaq Gold® DNA polymerase (Applied Biosystems; Foster City, Calif.). The amount of template DNA was as indicated below for each experiment. After denaturation at 95° C. for 10 min, PCPE was performed for 25-50 cycles, each cycle being 30 sec at 95° C., 120 sec at 58° C., 60 sec at 54° C. and 60 sec at 72° C. A final extension of 5 min at 72° C. was also used.

After PCPE, the extension products (single-strand DNA fragments) were captured using streptavidin-coated magnetic beads (Dynal Biotech; Lake Success, N.Y.). Twenty-five μl of extension products were mixed with an equal volume of magnetic beads in B&W buffer (10 mM Tris-HCl, pH 7.5, 1 mM EDTA, 2.0 M NaCl) and incubated at room temperature for 1-3 hours. Thereafter, the supernatants were removed, followed by washing the beads with 200 μl of 0.1 M NaOH for 5 min, and two additional washes using water.

The purified beads, containing the single-stranded DNA fragments, were resuspended in 5 μl of water. These DNA fragments were the templates for the fluorescence-based PCR reaction. The PCR mixture contained 1X PCR buffer, 0.2 mM each of dCTP, dTTP, dATP and dGTP nucleoside triphosphates, 2 mM of MgCl₂, 0.1 μM of the forward and reverse primers and 0.5 units of Taq Gold® polymerase. After denaturation at 95° C. for 10 min, PCR (25 μl) was performed for 42 cycles, each cycle being 30 sec at 95° C., 30 sec at 54° C., and 30 sec at 72° C. A final extension of 5 min at 72° C. was used. The primers for TGF-β RII microsatellites were, 5′-GAAGATGCTGCTTCTCCAA-3′ (SEQ ID NO. 13) and 5′-D4-ATCAGAGCTACAGGAACAC-3′ (SEQ ID NO. 14).

The fluorescently-labeled products of the PCR were analyzed by size using a CEQ8000 sequencer (Beckman Coulter; Fullerton , Calif.). The diagrams in the figures show the length of the DNA fragment analyzed on the x-axis, and the amount of the particular fragment on the y-axis.

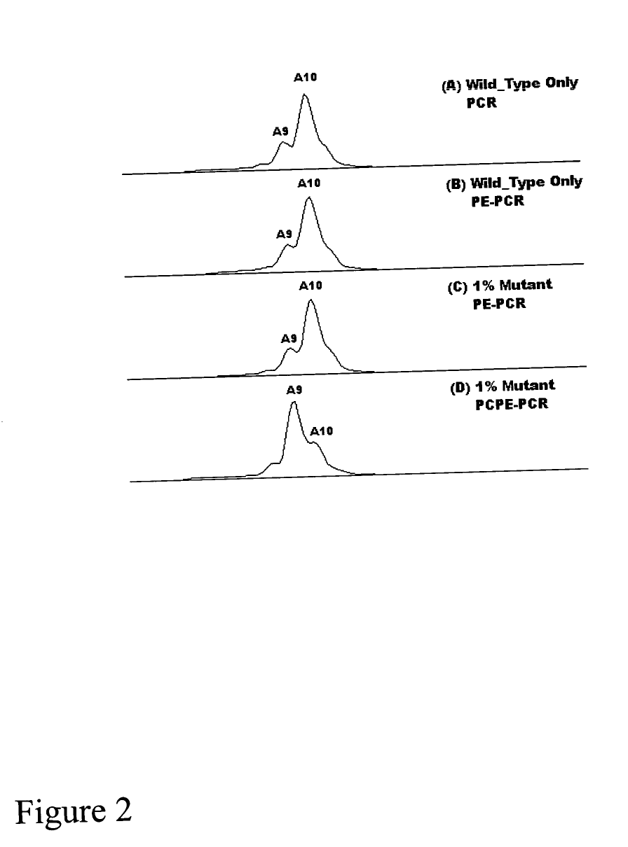

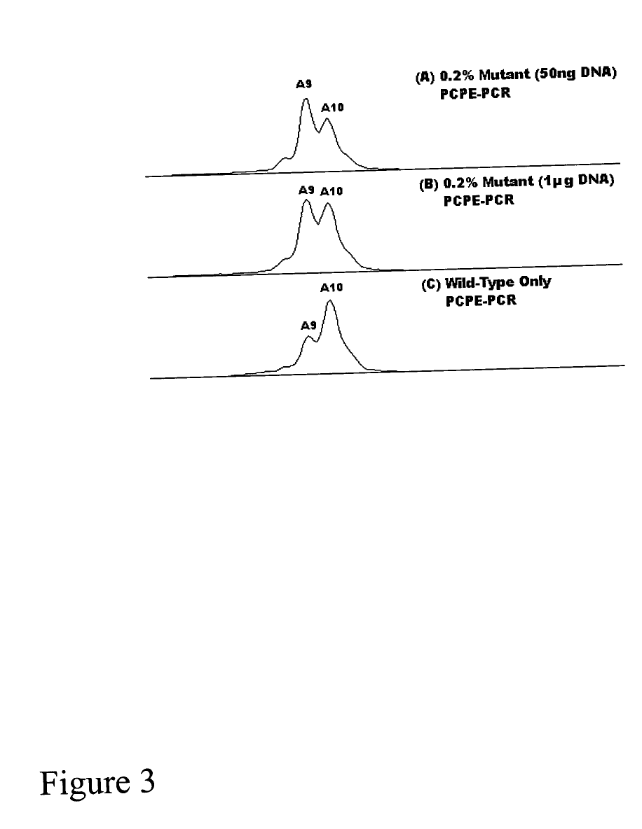

The results from this study are shown in FIGS. 2 and 3. In FIG. 2A, 50 ng of wild-type DNA was used in PCR. No primer extension was used. The data show that, even in the absence of mutant microsatellite (i.e., (A)₉) in the template, some (A)₉ (SEQ ID NO. 8) is generated by the PCR reaction. This (A)₉ (SEQ ID NO. 8) is the result of “PCR slippage” which occurs when errors are made by the polymerase in copying the template. The result of PCR slippage, is one or more PCR products with a deletion. The amount of the (A)₉ (SEQ ID NO. 8) product in this experiment was less than 50% of the (A)₁₀ (SEQ ID NO. 7) product. Generally, we have found that when PCPE-PCR is used, the amount of mutant product is greater than 80% of wild-type product, when mutant microsatellites were present in the template DNA used for the PCR. However, as is shown in FIG. 2C below, in the absence of PCPE-PCR, presence of mutant microsatellite in the template does not ensure production of mutant product that is 80% greater than wild-type product.

In FIG. 2B, 50 ng of wild-type DNA was used in primer extension (PE) in the absence of the blocking probe. The single-stranded DNA products (extension products) were purified using streptavidin-coated magnetic beads, as described above, and then used in PCR. The results are similar to those shown in FIG. 2A, in that the amount of (A)₉ (SEQ ID NO. 8) product produced was less than 50% of the amount of (A)₁₀ (SEQ ID NO. 7) product. These results are consistent with generation of a mutant microsatellite as a result of PCR slippage.

In FIG. 2C, 0.5 ng of mutant DNA mixed with 50 ng of wild-type DNA (1% mutant microsatellites) was used in PE (i.e., no blocking probe), single-stranded extension products were purified and were then used in PCR. The data, in the absence of PCPE, are very similar to that in FIGS. 2A and 2B in that the amount of the (A)₉ (SEQ ID NO. 8) product produced was less than 50% of the (A)₁₀ (SEQ ID NO. 7) product amount.

In FIG. 2D, 0.5 ng of mutant DNA mixed with 50 ng of wild-type DNA (1% mutant microsatellites) was used in PCPE (i.e., blocking probe was used), single-stranded extension products were purified and were then used in PCR. The data show, that when PCPE was used, there was a significant enrichment of the (A)₉ (SEQ ID NO. 8) product as compared to the (A)₁₀ product. Here, the amount of (A)₉ (SEQ ID NO. 8) product is significantly greater than the amount of (A)₁₀ (SEQ ID NO. 7) product.

In FIG. 3A, 0.1 ng of mutant DNA mixed with 50 ng of wild-type DNA (0.2% mutant microsatellites) was used in PCPE-PCR, as in FIG. 2D. The data show that, as in FIG. 2D, the amount of (A)₉ (SEQ ID NO. 8) product was significantly greater that the amount of (A)₁₀ (SEQ ID NO. 7) product. The data show that 0.2% of mutant microsatellites were detectable by the PCPE-PCR method.

In FIG. 3B, 2 ng of mutant DNA mixed with 1 μg of wild-type DNA (0.2% mutant microsatellites) was used in PCPE-PCR. The data show that the amounts of the (A)₉ (SEQ ID NO. 8) and (A)₁₀ (SEQ ID NO. 7) products are similar but, because the amount of mutant product is greater than 80% of wild-type product, the method successfully detected the mutant microsatellites. Comparison of the experiments in FIG. 3A (50 ng total DNA, 0.2% mutant) with FIG. 3B (1 μg total DNA, 0.2% mutant) shows that the PCPR-PCR method has a large dynamic range of input DNA.

The data shown in FIG. 3C are from a negative-control experiment. A DNA sample containing only wild-type DNA and no mutant DNA was used in PCPE-PCR. The results show the presence of some mutant (A)₉ (SEQ ID NO. 8) product, which is consistent with PCR slippage, but the amount of this product was less than 50% the amount of the wild-type (A)₁₀(SEQ ID NO. 7) product. Again using the threshold that mutant product levels greater than 80% of wild-type product levels indicates mutant in the input DNA, the results of this experiment indicate no mutant DNA in the input sample

### Example 4

### PCPE-PCR Applied to Long Microsatellite Sequences

PCPE-PCR was next used to detect mutations in long microsatellite sequences. Herein, long microsatellite sequences contain 20 or more repeats, and typically contain multiple nucleotide bases. In these studies, the BAT26 microsatellite was used. BAT26 in its wild-type form contains (T)₅ . . . (A)₂₆ (the dots indicate nonrepetitive nucleotides). BAT26 is an excellent marker for MSI-H colorectal cancer. BAT26 typically contracts 10 or more bases in colorectal cancer, but often less than 10 bases in adenoma. DNA from the HEC1A cell line was used in these studies. In HEC1A, one allele of BAT26 is contracted approximately 12 nucleotide bases (herein, “large-contracted BAT26”), while the other allele is contracted about 6 nucleotide bases (herein, “small-contracted” BAT26). Using DNA from this cell line, it was possible to evaluate both deletions within the BAT26 microsatellite.

PCPE was carried out as described in Example 3 except that the blocking probe for BAT26, as described in Example 2 for BAT26, was used. Additionally, the extension primer was 5′-Biotin-TGCAGTTTCATCACTGTCTGC-3′ (SEQ ID NO. 5) and the PCPE was performed for 25-50 cycles, each cycle being 30 sec at 95° C., 120 sec at 68° C., 60 sec at 62° C. and 60 sec at 72° C. A final extension of 5 min at 72° C. was used.

After PCPE, the formed single-strand DNA fragments (extension products) were captured using streptavidin-coated magnetic beads (Dynal Biotech; Lake Success, N.Y.), as described in Example 3.

Fluorescence-based PCR was carried out as described in Example 3 except that the primers for BAT26 microsatellites were, 5′-D4-ATTGGATATTGCAGCAGTC-3′ (SEQ ID NO. 10) and 5′-AACCAATCAACATTTTTAACCC-3′ (SEQ ID NO. 11).

The fluorescently-labeled products of the PCR were analyzed by size using a CEQ8000 sequencer (Beckman Coulter; Fullerton, Calif.). The diagrams in the figures show the length of the DNA fragment analyzed on the x-axis, and the amount of the particular fragment on the y-axis.

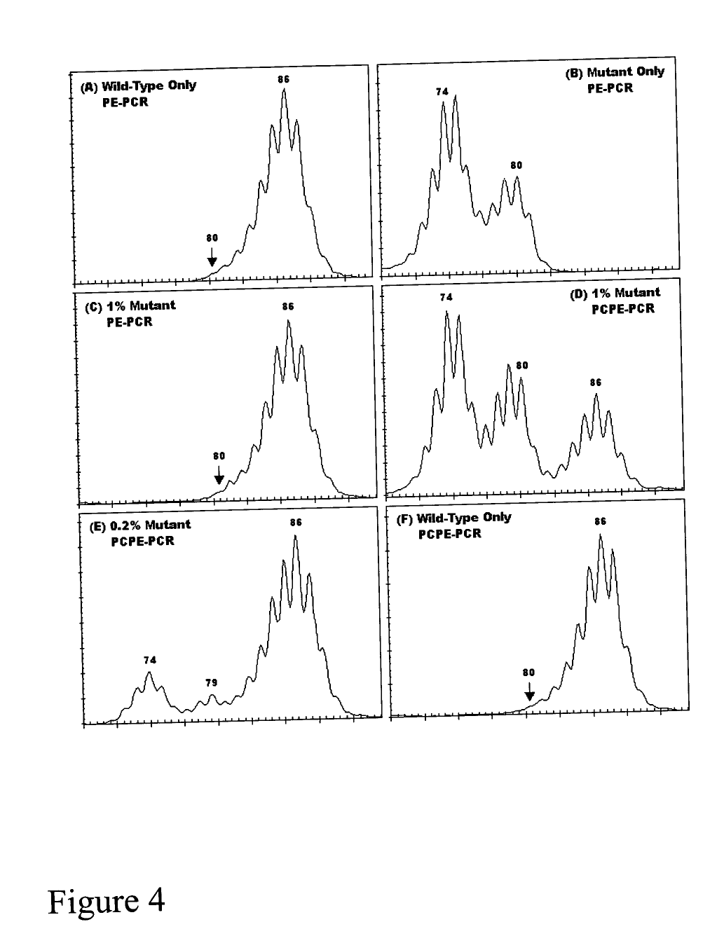

The results of this study are shown in FIG. 4. FIGS. 4A and 4B show the results of primer extension (PE) in the absence of blocking probe, purification of the resulting single-stranded products, and use of the single-stranded products as templates in PCR, for wild-type DNA alone (FIG. 4A) or for mutant DNA alone (FIG. 4B). The data show that, for wild-type BAT26, the major PCR product is 86 nucleotides in length (FIG. 4A). For mutant BAT26, the major peak for large-contracted BAT26 (i.e., the BAT26 allele missing 12 nucleotide bases) is 74 nucleotides in length. The major peaks for small-contracted BAT26 (i.e., the BAT26 allele missing 6 nucleotide bases) are 79 and 80 (FIG. 4B). The results show a distribution of minor peaks around the main peak for each of the three BAT26 alleles.

FIG. 4C shows the results from using 0.5 ng of mutant DNA mixed with 50 ng of wild-type DNA (1% mutant microsatellites) as templates in primer extension in the absence of blocking probe (PE), purification of the resulting single-stranded extension products, and use of the single-stranded products as templates in PCR. The resulting pattern of fragments (FIG. 4C) is similar to that shown in FIG. 4A, where the input template DNA contained no mutant DNA. In the absence of the blocking probe in the PE reaction, therefore, 1% mutant microsatellites was undetectable.

FIG. 4D shows the results from using the identical input DNA as was used in the FIG. 4C experiment (1% mutant DNA). However, the results in FIG. 4D were obtained with use of the blocking probe in the PE step. The results (FIG. 4D) show that, in contrast to the inability to detect mutant DNA in the absence of blocking probe (FIG. 4C), with blocking probe (FIG. 4D), both the large-contracted and small-contracted BAT26 alleles were clearly detected. FIG. 4E shows that as little as 0.2% mutant DNA can be detected using the blocking probe in PCPE-PCR.

FIG. 4F shows results from a negative-control experiment. In this experiment, a DNA sample containing only wild-type DNA and no mutant DNA was used in PCPE-PCR. The results very little, if any, of PCR products attributable to presence of large-contracted or small-contracted BAT26 DNA in the input sample.

What is claimed is:

1. A method for detecting a mutant polynucleotide in a mixture of mutant polynucleotides, wild-type polynucleotides and unrelated polynucleotides, comprising the steps of:
a) selecting an extension primer complementary to a first target sequence in the mutant and wild-type polynucleotides;
b) selecting a probe complementary to a second target sequence in the wild-type polynucleotides but not in the mutant polynucleotides,
wherein the second target sequence is located 3′ of the first target sequence on the same polynucleotide strand;
c) contacting the polynucleotides in the mixture with the probe under conditions where the probe preferentially anneals to the second target sequence rather than to a corresponding sequence in the mutant polynucleotides;
d) contacting the polynucleotides in the mixture with the extension primer under conditions where the primer anneals to the first target sequence;
e) contacting the polynucleotides in the mixture with a DNA polymerase and nucleoside triphosphates under conditions where the extension primers are extended by polynucleotide synthesis, using the mutant and wild-type polynucleotides as templates, to produce extension products,
wherein polynucleotide synthesis that uses the wild-type polynucleotides as templates is blocked by the probe;
e) isolating the extension products from the mixture; and
f) amplifying the extension products produced from mutant polynucleotide templates preferentially over amplifying extension products produced from wild-type polynucleotide templates using the polymerase chain reaction (PCR).

2. The method of claim 1, wherein the mutant polynucleotides contain deletion mutations, insertion mutations, substitution mutations or a combination of deletion, insertion and substitution mutations, as compared to the wild-type polynucleotides.

3. The method of claim 1, wherein the mutant and wild-type polynucleotides are isolated from the mixture of mutant polynucleotides, wild-type polynucleotides and unrelated polynucleotides before the step of contacting the polynucleotides with the probe.

4. The method of claim 3, wherein the mutant and wild-type polynucleotides are isolated using a sequence specific hybrid capture method.

5. The method of claim 1, wherein the extension primer has one or more attached biotin molecules.

6. The method of claim 1, wherein the probe is a peptide nucleic acid

7. The method of claim 1, wherein the probe is an oligonucleotide.

8. The method of claim 7, wherein at least part of the oligonucleotide has a phosphorothioated backbone.

9. The method of claim 7, wherein the oligonucleotide has a 5′ end and a 3′ end and the 3′ end is modified such that it cannot be extended by polynucleotide synthesis.

10. The method of claim 9, wherein the nucleotide at the 3′ end of the oligonucleotide is phosphorylated.

11. The method of claim 1, wherein the probe is a locked nucleic acid, modified oligonucleotide or oligonucleotide analogue.

12. The method of claim 1, wherein:
a) there is a first Tₘ for annealing of the extension primer to the first target sequence;
b) there is a second Tₘ for annealing of the probe to the second target sequence; and
c) there is a third Tₘ for annealing of the probe to the mutant polynucleotides,
wherein the second Tₘ is higher than the first Tₘ; and
wherein the first Tₘ is higher than the third Tₘ.

13. The method of claim 1, wherein the first target sequence and the second target sequence overlap.

14. The method of claim 1, wherein polynucleotide synthesis preferentially extends extension primers that have annealed to the first target sequence in mutant polynucleotides.

15. The method of claim 1, wherein the extension products are isolated from the mixture by a solid phase extraction method.

16. The method of claim 1, wherein the extension products from mutant polynucleotides as templates are preferentially isolated from the mixture.

17. The method of claim 1, wherein the PCR uses;
a) a first PCR primer that is complementary to a nucleotide sequence present in the 3′ end of a long extension product, but not present in a short extension product; and
b) a second PCR primer that is identical to a nucleotide sequence present in both the long and short extension products.

18. The method of claim 1, comprising the additional step of analyzing the amplified extension products from the PCR.

19. A method for detecting a mutant microsatellite in a mixture of mutant microsatellites and wild-type microsatellites, in a sample of genome DNA from an individual, comprising:
a) contacting a probe with the microsatellites in the mixture under conditions where the probe preferentially anneals to a second target region in the wild-type microsatellites as compared to a corresponding region in the mutant microsatellites,
wherein the corresponding region in the mutant microsatellites differs in nucleotide sequence from the second target region in the wild-type microsatellites;
b) contacting an extension primer with the microsatellites in the mixture under conditions where the extension primer anneals to a first target region in both the mutant and wild-type microsatellites, that is on the same strand as and located 5′ of the second target region in the wild-type satellites, and is on the same strand as and located 5′ of the corresponding sequence in the mutant microsatellites;
c) contacting a polymerase and nucleoside triphosphates with the microsatellites in the mixture under conditions where polynucleotide synthesis extends the extension primers using the microsatellites as templates to produce extension products,
wherein polynucleotide synthesis that uses wild-type microsatellites as templates is preferentially blocked by the probe as compared to polynucleotide synthesis that uses mutant microsatellites as templates;
d) isolating the extension products from the mixture;
e) amplifying the extension products by PCR to produce PCR products,
wherein the extension products that used mutant microsatellites as templates are preferentially amplified as compared to extension products that used wild-type microsatellites as templates;
f) analyzing the extension products amplified by the PCR.

20. The method of claim 19, wherein the microsatellites are TGF-β RII (A)10 or BAT26 microsatellites.

21. The method of claim 19, wherein the microsatellites are NR-21 microsatellites.

22. The method of claim 19, wherein the genome DNA is from a stool or blood sample.

23. The method of claim 19, wherein a multiplexed assay is used to simultaneously detect two or more mutant microsatellites.

24. The method of claim 19 wherein a multiplexed assay is used to simultaneously detect mutant TGF-β RII (A)10 and BAT26 microsatellites.

25. A method for selectively amplifying a mutant polynucleotide, if any, in a mixture of wild-type polynucleotides and unrelated polynucleotides, both the mutant polynucleotide and the wild-type polynucleotides having the same first target sequence, the wild-type polynucleotide having a second target sequence not present in the mutant polynucleotide, at least part of the second target sequence being located 3′ of the first target sequence on the same polynucleotide strand of the wild-type polynucleotide, the process comprising:
a) contacting the mixture with a probe complementary to the second target sequence to preferentially anneal the probe to the second target sequence of the wild-type polynucleotide rather than to a corresponding sequence in the mutant polynucleotide;
b) contacting the mixture with an extension primer complementary to the first target sequence in both the mutant and wild-type polynucleotides to anneal the primer to the first target sequence in both polynucleotides; and
c) contacting the mixture with a DNA polymerase and nucleoside triphosphates to extend the extension primers annealed to the polynucleotides by polynucleotide synthesis,
the probe annealed to the wild-type polynucleotide limiting polynucleotide synthesis of the extension primer annealed to the wild-type polynucleotide.

26. The process of claim 25, further comprising:
a) isolating extended extension primers from the mixture; and
b) contacting the extended extension primers with a first and second PCR primer, a PCR polymerase and nucleoside triphosphates to amplify the extended extension primers by PCR,
the PCR preferentially amplifying extended extension primers from extension primers annealed to mutant polynucleotides.

27. The process of claim 25, further comprising determining the size and abundance of amplified extended extension primers from the PCR.
